(19) United States
(12) Patent Application Publication
(10) Pub. No.: US 2010/0130981 A1
(43) Pub. Date: May 27, 2010

(54) WRIST PREPARATION SYSTEM AND METHOD

(75) Inventor: Mark I. Richards, Austin, TX (US)

Correspondence Address: FITCH EVEN TABIN & FLANNERY, 120 SOUTH LASALLE STREET, SUITE 1600, CHICAGO, IL, 60603-3406, US

(73) Assignee: ASCENSION ORTHOPEDICS, INC., Austin, TX (US)

(21) Appl. No.: 12/525,521

(22) PCT Filed: Jan. 30, 2008
(86) PCT No.: PCT/US08/52491

§ 371 Date: Nov. 11, 2009

Related U.S. Application Data
(60) Provisional application No. 60/888,009, filed on Feb. 2, 2007.

(51) Int. Cl. A61F 5/56; A61F 2/42

(52) U.S. Cl. 606/87; 623/21.12

(57) ABSTRACT

Surgical instruments and methods for the resection of bones for a total wrist replacement, including a kit of dual broaches/radial and carpal implants. The instrument affords the surgeon the opportunity to set a precise, single window that establishes two parallel cutting planes, defined by two pairs of upper and lower bars 10 and 11 that are found in two relatively movable blocks 1 and 4, along which resection of the bones will take place prior to the occurrence of any resection. The employment of the same instrument to prepare either a right or left wrist and different sized dual broach/radial and carpal trial implants simplifies preparation and reduces operating room time for such a total wrist replacement.

This application claims priority from U.S. Provisional Application Ser. No. 60/888,009, filed Feb. 2, 2007, the disclosure of which is incorporated herein by reference.

The invention relates to surgical instruments and to methods for using same to resect bones for a joint replacement, and more particularly to instruments useful for bone preparation for a total wrist replacement and methods for using such instruments in resection of the bones at the wrist.

### BACKGROUND OF THE INVENTION

Various wrist prostheses have been developed in the past several decades that allow total wrist replacement by resecting the radius and various carpal bones and then implanting components that provide an articulating joint that allows for movement in different planes. Typical of these total wrist replacement prostheses are those shown in U.S. Pat. Nos. 4,063,314, 4,180,871, 5,314,485, 5,702,470 and 6,746,486, as well as U.S. published application 2006/0161260. Devices have also been developed for cutting the radial and carpal bones to receive such a total wrist prosthesis, such as those shown in U.S. Pat. No. 6,827,741.

Because of the frequency with which orthopedic surgeons are employing such more advanced total wrist prostheses, there are continuing efforts to improve bone preparation techniques and instrumentation to support such techniques.

### SUMMARY OF THE INVENTION

The invention provides an improved surgical instrument for facilitating the resection of the radius and the carpal bones to prepare the wrist of a patient for the implantation of a total wrist replacement prosthesis. The instrument is adaptable and versatile enough that a single instrument can be effectively used on both the right and left wrists of patients of varying sizes in order to carry out the osteotomies suitable for the implantation of implants of varying sizes, as appropriate with respect to a particular patient.

More particularly, a surgical instrument is provided which is attached dorsally to the patient's wrist and then adjusted by the surgeon to locate parallel cutting guides at spaced apart locations aligned with the radius and the carpal bones so as to guide osteotomies that will effect the desired extent of resection of these bones. The initial attachment of the surgical instrument is preferably via a plurality of elongated pins or K-wires, some of which can optionally also be used to subsequently install an intermedullary placement guide upon the resected radius that facilitates insertion of a guide rod into the intramedullary (IM) canal of the radius; this rod is then utilized to guide an appropriately sized radial broach/trial implant therealong so that a stem portion of the implant is received in the IM canal and a head thereof lies flush against the surface of the resected radius. Another guide rod is inserted into the TM canal of one of the resected carpal bones, usually the capitate, and an appropriately sized carpal broach/trial implant is similarly inserted into the IM canal along such guide rod.

Following the mating of a trial articular element to the trial carpal implant, a trial reduction is carried out to assess range of motion (ROM) and stability in flexion and extension. If satisfactory, the trial articular element and the two trial implants are removed and replaced by components of a total wrist implant, such as that shown in the above-mentioned '260 published U.S. application.

In one particular aspect, the invention provides a surgical instrument for mounting dorsal of a patient's wrist to guide resection of the radius and the carpal bone complex during preparation for implanting a radial component and a carpal component as part of a total wrist replacement, which surgical instrument comprises (a) a radial osteotomy block having a cutting guide surface, (b) a carpal osteotomy block having a cutting guide surface, (c) means linking said osteotomy blocks in generally planar orientation so that said blocks can move longitudinally relative to each other to alter the separation distance between said cutting guide surfaces of said blocks, (d) means for attaching said instrument to the radius and a carpal bone of a patient in a manner so said osteotomy blocks remain longitudinally movable relative to these bones, (e) means for adjusting the longitudinal distance between said cutting guide surfaces to facilitate osteotomy of the patient's wrist to the desired degree, and (f) means for stably attaching said blocks to bones at the wrist of a patient with said cutting guide surfaces spaced the desired distance apart.

In another particular aspect, the invention provides a surgical kit for preparing the wrist of a patient for implantation of a total wrist prosthesis, which kit comprises (a) a surgical instrument for mounting dorsal of a patient's wrist, which instrument includes a radial osteotomy block having a cutting guide surface, a carpal osteotomy block having a cutting guide surface, means linking said osteotomy blocks in generally planar orientation so that said blocks can move longitudinally relative to each other to alter the separation distance between said cutting guide surfaces of said blocks, means for attaching said instrument to the radius and to one carpal bone of a patient in a manner so that said osteotomy blocks can be moved distally-proximally relative to said bones, means for adjusting the longitudinal distance between said cutting guide surfaces to facilitate osteotomy of the patient's wrist to the desired degree while said instrument is so attached, and means for stably affixing said instrument to bones at the wrist of a patient with said cutting guide surfaces are spaced the desired distance apart; (b) a plurality of radial broach/trial implants each having a head with a concave surface and a broaching stem, the heads of which radial implants range in size; (c) a plurality of carpal broach/trial implants each having a head with a protruding interconnector and a broaching stem, the heads of which carpal implants range in size; and (d)at least one articular insert for connection with said carpal broach/trial implant interconnector, which insert has a convex surface of a curvature matched to articulate with said concave surface of one said radial broach/trial implant.

In a further particular aspect, the invention provides a method for the implantation of a total wrist replacement, which method comprises the steps of: (a) attaching a surgical instrument dorsal of the patient's wrist by initially attaching said instrument to the radius and to a carpal bone, said instrument comprising a radial osteotomy block and a carpal osteotomy block which are longitudinally movable relative to each other, each of which blocks has a cutting guide surface; (b) adjusting said radial osteotomy block proximally/distally relative to the distal end of the radius and securing said radial osteotomy block in said adjusted location; (c) positioning said carpal osteotomy block at a desired location where its cutting guide surface is at a desired distance from the cutting guide surface of said radial block and is aligned with bones of the carpal bone complex to be resected, and securing said carpal block in such location; (d) respectively cutting the radius and bones of the carpal bone complex along planes defined by said two cutting guide surfaces of said radial and carpal osteotomy blocks; (e) removing said surgical instrument from the wrist; and (f) broaching the resected radius and one resected carpal bone preparatory to installing a total wrist replacement.

In yet another particular aspect, the invention provides a method for the implantation of a total wrist replacement, which method comprises the steps of (a) cutting the radius and bones in the carpal bone complex of the patient's wrist along two spaced apart planes to carry out the desired resection; (b) broaching the radius through the resected end thereof using a combined radial broach/trial implant which is allowed to remain therein; (c) broaching a resected carpal bone using a combined carpal broach/trial implant which is allowed to remain therein; (d) attaching a trial articular element to said carpal broach/trial implant; (e) performing a trial reduction to assess range of motion (ROM); (f) removing said trial element and trial implants; and (g) installing a radial component, a carpal component and an articular element to provide a total wrist replacement.

### BRIEF DESCRIPTION OF THE DRAWINGS

FIG. 1 is an exploded perspective view showing a surgical instrument that is designed to facilitate osteotomies to prepare a patient for the implantation of a total wrist prosthesis.

FIG. 2 is a perspective view showing the assembled surgical instrument placed dorsal of a skeletal structure representing the right hand of a patient.

FIG. 2a is a perspective view similar to FIG. 2 showing an elongated pin inserted through the instrument into the radius.

FIG. 3 is a side view, enlarged in size, of the instrument and skeletal structure shown in FIG. 2 showing the two elongated pins that are used to attach the instrument to the radius and to the third metacarpal of the patient.

FIG. 4 is a top view of the arrangement shown in FIG. 3.

FIG. 5 is a perspective view similar to FIG. 2 showing two additional elongated pins implanted in the radius once the desired sizing has been determined.

FIG. 6 is an enlarged fragmentary view focused on the surgical instrument showing the distal end of the radius and portions of the carpal bone complex as viewed through a window in the instrument once the surgeon has determined its desired placement adjacent the distal end of the radius.

FIG. 7 is a view similar to FIG. 5 showing two additional elongated pins that were implanted in carpal bones once the desired sizing window has been determined.

FIG. 8 is a view of the skeletal structure of the patient following resection of the bones prior to removal of the surgical instrument and then insertion of a radial IM placement guide using the three elongated pins which were earlier inserted into the radius to secure the radial osteotomy block of the surgical instrument.

FIG. 9 is a view of the skeletal arrangement shown in FIG. 8, with the three pins now removed from the carpal bones and with the wrist shown flexed, as an elongated guide rod is driven through the placement guide into the intramedullary canal of the radius.

FIG. 10 is a schematic view showing the broaching of the IM canal of the radius using a radial broach/trial implant that slides along the previously implanted, elongated guide rod.

FIG. 11 is a view similar to FIGS. 9 and 10 showing the trial implant seated in the radius with its head flush with the resected surface thereof, with the elongated guide rod removed and with an elongated guide rod inserted into the IM canal of the resected capitate.

FIG. 12 is a schematic view showing a carpal broach/trial implant being driven into the IM canal of the capitate.

FIG. 13 is a view similar to FIGS. 10-12 showing the carpal broach/trial implant seated against the resected carpal bones and with an articular insert placed over a connecting post or trunnion that protrudes from its exposed surface that faces the radius.

FIG. 14 is a view similar to FIG. 13 after the articular insert has been rotated 90° to rotatably secure it to the dual purpose carpal implant and after the wrist has been returned to extension position to assess ROM and stability.

FIG. 15 is a perspective view, similar to FIG. 12, showing the skeletal structure after the trial insert and two trial implants have been removed, after a radial component has been inserted into the resected end of the radius in place of the trial implant and as the stem of a carpal base plate component is being inserted into the broached IM canal of the capitate.

FIG. 16 is an enlarged view, similar to FIG. 15, showing the carpal component in place with a bone screw being inserted into one of the carpal bones through one of the two openings in the base plate that flank the stem and the center attachment post.

FIG. 17 is a view similar to FIG. 16 showing the articular insert after it is inserted over the attachment post or trunnion of the carpal implant, but prior to its rotation of 90° which secures it in rotative attachment.

FIG. 18 is a perspective view showing the skeletal structure with the total wrist prosthesis in place.

FIG. 19 is an exploded perspective view of a representative prior art total wrist prosthesis.

### DETAILED DESCRIPTION OF THE PREFERRED EMBODIMENTS

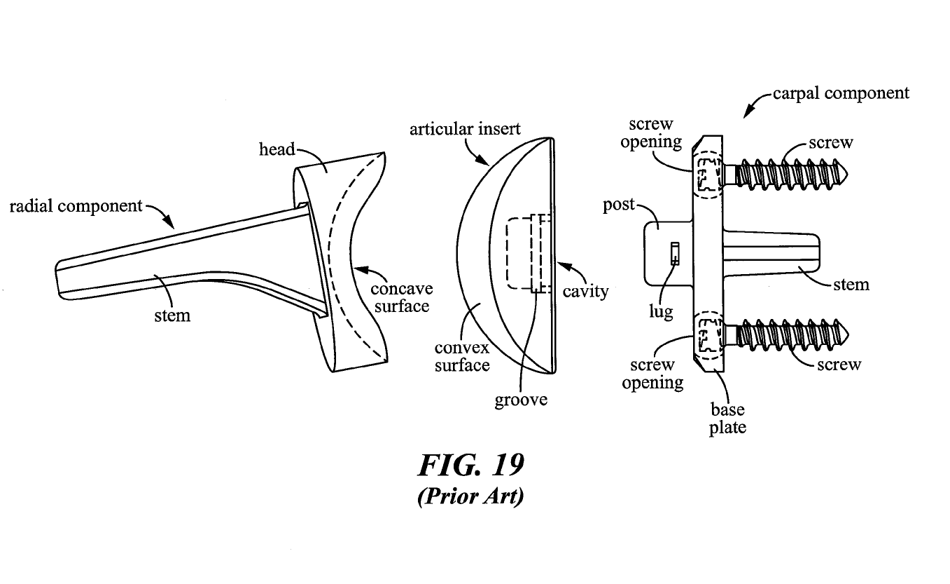

Disclosed in the drawings is a surgical instrument which is designed to assist a surgeon in resecting bones at the wrist of a patient to prepare for the implantation of a total wrist replacement prosthesis. It should be understood that the instrument and the methods disclosed are suitable for implantation of a variety of total wrist replacement prostheses; however, for purposes of illustration, the description variously makes reference to a total wrist prosthesis of the type illustrated in U.S. Patent Publication No. 2006/0161260 (Jul. 20, 2006), the disclosure of which is incorporated herein by reference. FIG. 19 reproduces a FIGURE from that publication. This prosthesis comprises a radial component having a stem and a concave articulating surface, a carpal component having a stem and a central post (the two of which extend in the opposite directions), and an articular element or insert having a cavity to receive the post and a convex surface to articulate against the concave surface of the radial component.

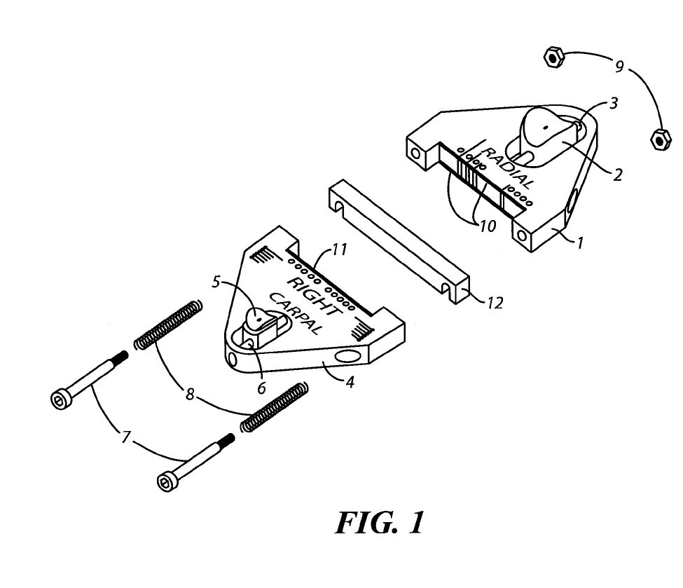

The surgical instrument is designed to be attached dorsally to the wrist of the patient after incision of the skin and its folding back to expose the distal end of the radius and the associated carpal bone complex, extending as far as the third metacarpal. As best seen in FIG. 1, the surgical instrument includes a radial osteotomy block 1 and a carpal osteotomy block 4; they have generally flat, parallel upper and lower surfaces and are linked or interconnected with each other so as to permit the blocks to be uniformly separated from each other by relative movement along a longitudinal axis. Interconnection can be established in any suitable manner, but it is preferably achieved via a pair of threaded rods or shoulder bolts 7 having threaded ends and elongated, smooth shoulders which accommodate compression springs 8 disposed about the shoulder regions. The bolts extend through parallel passageways provided in lateral regions of the carpal osteotomy block 4. The springs 8 are confined within the passageways, seating against heads at the distal ends of the bolts 7. The threaded proximal ends reside in aligned passageways in the radial osteotomy block 1, which passageways are counterbored at the proximal ends thereof to accommodate a pair of hex nuts 9. The springs 8 are sized to maintain facing flat surfaces of the two blocks in face-to-face contact with each other but to allow relative movement away from each other.

The abutting surfaces constitute spaced apart, generally square surfaces located at the respective sides of each block which squares are interrupted by cutout regions that each define a portion of a window of the instrument and extend uniformly to respective cutting guide surfaces. The two cutting guide surfaces lie in two spaced-apart planes that are parallel to each other. Each plane is defined by a pair of upper and lower rectilinear bars 10 and 11 of hard metal that are located at the upper and lower surfaces of the respective blocks and that are spaced apart from each other. The rectilinear bars 10, 11 can be of circular, oval, square or other suitable cross-section. Although other constructions could be used, the blocks 1 and 4 are conveniently made by molding such radiopaque metal bars within a radiolucent polymeric material that is sterilizable, such as an acetyl (POM) resin or an acetyl copolymer resin. The radial osteotomy block 1 preferably also contains other embedded radiopaque elements as described hereinafter.

Each of the blocks 1 and 4 is formed with a central elongated aperture, preferably oval in shape, that extends transversely completely therethrough, dorsal to volar; a longitudinally aligned shaft 3, 6 respectively resides in the apertures in the blocks 1 and 4. The shafts 3, 6 respectively carry slidable saddle parts 2, 5 that are relatively movable therealong longitudinally within the oval apertures. Although the two shafts 3, 6 are parallel to each other, they are not coaxial; they are laterally offset from each other and designed so that the instrument is suitable for use for the preparation of either a right wrist or left wrist of a patient. By simply inverting the instrument, the shafts 3 and 6 will respectively appropriately align with the centerlines of the radius and third metacarpal of either wrist of the patient whose wrist is to be repaired.

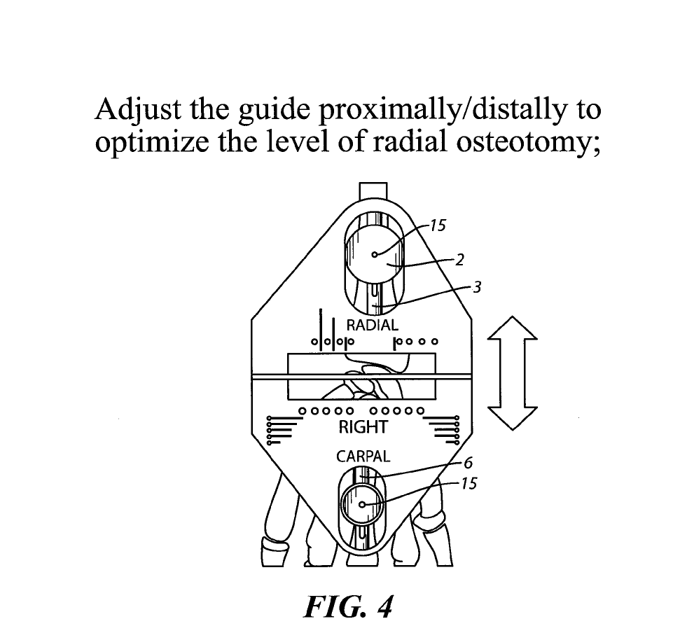

The saddle parts 2, 5 are slidably mounted on the shafts 3, 6 which shafts are conveniently of circular cross-section; the shafts can be inserted into the blocks 1, 4 through the end opposite from that which faces the other block passing through passageways in the saddle parts and secured in place. The saddle parts 2, 5 have flat side surfaces that are juxtaposed with the flat side surfaces of the oval apertures. Each of the parts 2 and 5 has a pair of diametrically opposed, upper and lower, saddle surfaces; these concave surfaces are shaped to respectively rest on the dorsal surface of the radius and the third metacarpal. For reasons to be described hereafter, each of the shafts 3, 6 is provided with an elongated slot (see FIG. 4) extending along its centerline; the slot extends volar to dorsal completely through each shaft. It allows the passage of a thin pin that will be inserted through a pinhole in the saddle part extending between the saddle surfaces and intersecting the centerline of the circular cross-section shaft 3 or 6; its elongation allows longitudinal movement of the remainder of the instrument relative thereto after the radial saddle part 2 is pinned to the radius.

Each block 1, 4 also has a line of spaced apart pinholes that also extend completely through the block, dorsal to volar, which line of pinholes is parallel to the cutting guide surfaces defined by the vertically aligned pairs of bars 10 and 11. The purpose of such lines of pinholes is explained hereinafter. The carpal osteotomy block 4 also has two longitudinally extending lines of openings or one elongated opening. One line is located near each lateral edge in general alignment with the centerlines of the passageways through which the shoulder bolts 7 extend. The socket-carrying heads of the shoulder bolts 7 can be observed through these openings for reasons explained hereinafter.

Shown in FIG. 1, in a position between the two osteotomy blocks 1 and 4, is a spacer body 12 which has a pair of grooves in its undersurface that allow it to be fit over the smooth cylindrical surfaces of the shoulder bolts 7. In a kit for preparation for a total wrist replacement, a plurality of such spacers of different widths is provided so that the surgeon can space the cutting surfaces 10, 11 of the two osteotomy blocks 1, 4 one of several precise longitudinal distances apart to facilitate the precise resection that is desired so as to prepare the wrist to accommodate an implant of an appropriate size to be selected from a variety of different size components that would normally be provided.

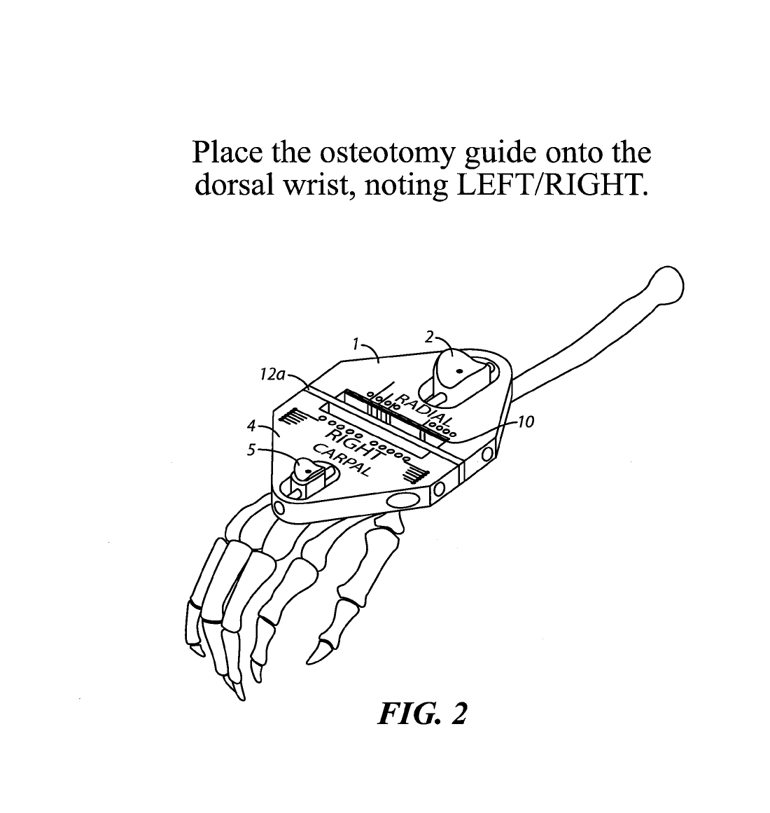

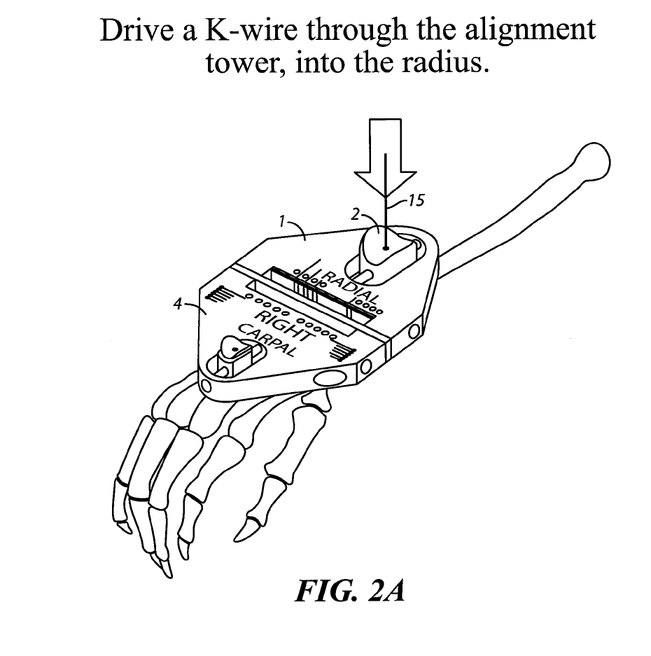

Methods for using the surgical instrument to resect the radius and the carpal bone complex to prepare it for implanting a total wrist replacement wrist prosthesis are now described. Following making incisions into the skin and folding back the flesh to expose the ends of the patient's radius and the carpal bone complex, extending to the third metacarpal, the surgical instrument is positioned dorsally of the wrist, as depicted in FIG. 2; in this illustration, placement is dorsally onto the right wrist of the patient. Alignment is such that the undersurface of the saddle part 2 of the radial osteotomy block 1 rests upon the dorsal surface of the radius, and the undersurface of the saddle part 5 of the carpal osteotomy block 4 rests on the dorsal surface of the third metacarpal. In the illustrations shown in FIGS. 2-7, a thin spacer block 12a (having a thickness less than the spacer 12 shown in FIG. 1) is located between the juxtaposed, flat, facing surfaces of the two osteotomy blocks 1 and 4. It should be clear that there either could be no spacer 12a initially included or a wider spacer block could be used.

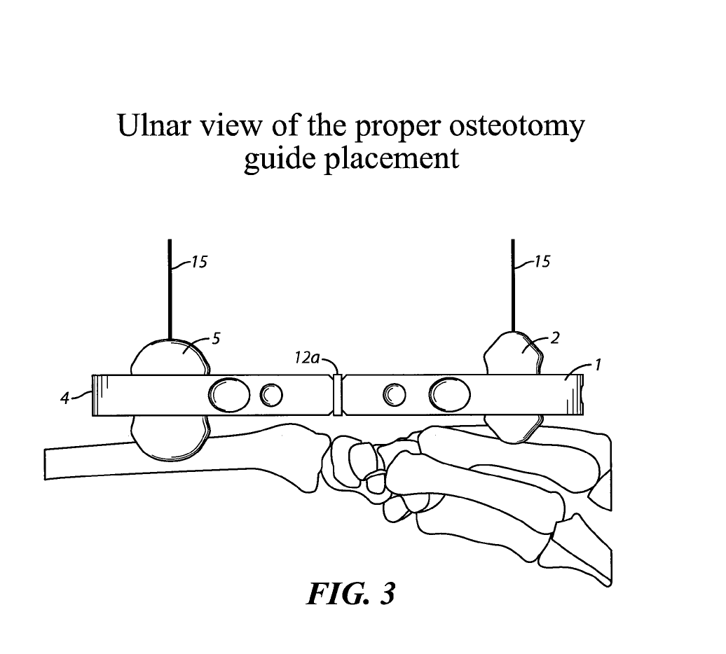

With the surgical instrument so generally positioned, thin wire pins or Kirschner wires (K-wires) 15 are then driven through the pinholes that extend through the centers of the saddle surfaces of the two saddle parts 2 and 5 so as to seat in the radius and the third metacarpal, respectively. FIG. 2A shows the K-wire 15 inserted into the radius. The side view shown in FIG. 3 illustrates this initial attachment of the surgical instrument to the right wrist of the patient with both K-wires 15 in place. This attachment maintains proper rotation of the hand and establishes a reference to the long axis of the third metacarpal and, accordingly, to the long axis of the capitate. This also constrains potential rotation of the surgical instrument relative to the parallel axes of the radius and third metacarpal.

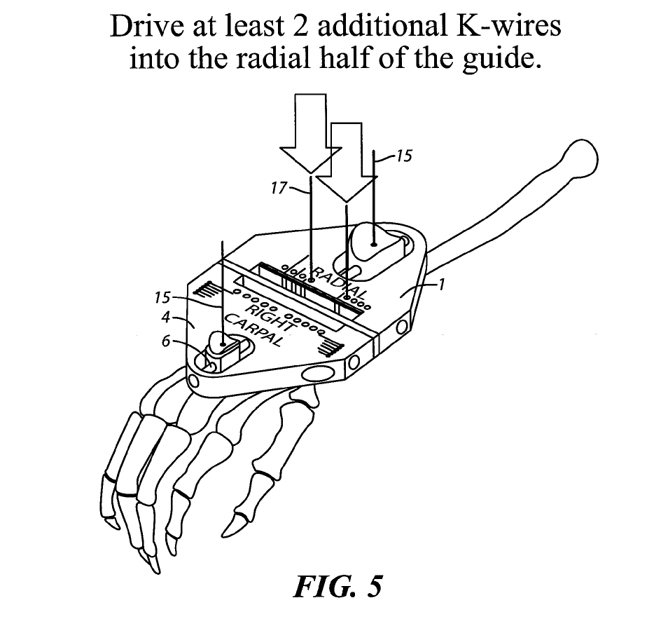

These two K-wires 15 pass respectively through the elongated slots in the two shafts 3 and 6 upon which the saddle parts 2 and 5 are slidably mounted, thus allowing the two osteotomy blocks 1, 4 to be moved longitudinally as a unit when the saddle parts are pinned to the respective bones. The shafts 3 and 6 slide through the anchored saddle parts 2 and 5 allowing the surgeon to initially position the cutting guide surface 10 of the radial osteotomy block 1 so it is aligned to guide a cut that will resect the distal end of the radius to the desired extent. One such prospective alignment of the cutting guide surface adjacent the end of the radius is depicted in FIG. 4. Once so positioned, at least two additional elongated pins or K-wires 17 are then driven into the dorsal surface of the radius through two selected pinholes in the row of spaced apart pinholes in the radial osteotomy block, as depicted in FIG. 5.

At this time, the size of radial component to be used is determined. Visual and fluoroscopic markings on and in the block 1 correspond with the radial and ulnar edges of the eminence of the different radial components that might be implanted. The appropriate-sized radial component will have a radial head that will be as large as possible, without overhanging the radial osteotomy. Determination of the selected size, e.g. S, M or L, is noted and referenced for the remainder of the procedure.

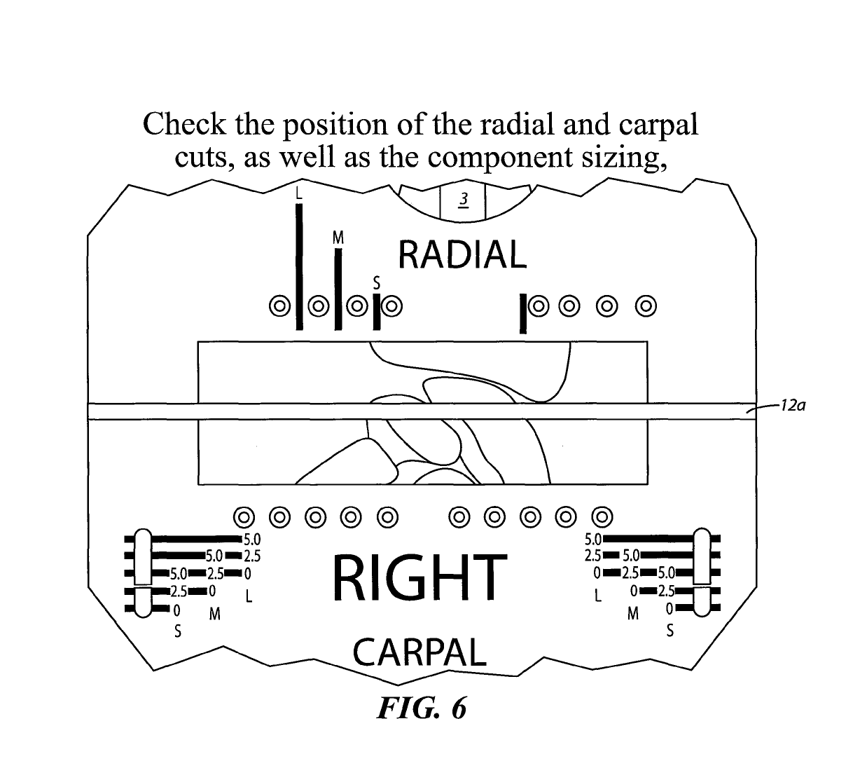

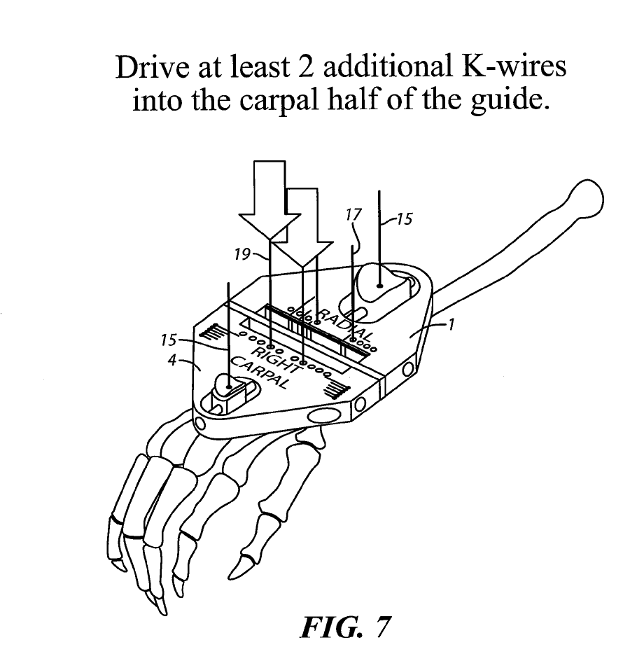

The surgeon then examines the alignment of the cutting guide surface defined by the bars 11 of the carpal osteotomy block 4 with respect to the carpal bone complex, see FIG. 6. Although the radial osteotomy block 1 is fixed at this point in time by the K-wires 16, the carpal osteotomy block 4 can be moved either toward or away from the radial block, i.e. either by removing the thin spacer 12a so the two blocks would abut, or by replacing the thin spacer 12a with a wider spacer 12 so the two osteotomy blocks (and their respective cutting guide surfaces) become spaced further apart from each other. When the desired spacing between the cutting guide surfaces is achieved by inclusion of spacer of appropriate width (or none at all), two additional K-wires 19 are driven into the carpal bone complex through selected pinholes in the line of openings in the block 4 which are in alignment with carpal bones of interest, as illustrated in FIG. 7.

With the surgical instrument thus secured and any optional spacer in place, the surgeon resects the bone using an oscillating saw or the like to cut along the pair of spaced rectilinear dorsal and volar bars 10 and 11 on each side of the window provided by the surgical instrument, being careful to keep the edge of the saw against both bars. Prior to actual cutting, the surgeon should examine the wrist and surgical instrument radiographically, i.e. fluoroscopically, to observe where the vertically spaced bars 10 and 11 that define the two cutting planes are precisely aligned with the bones, and checking again that the parallel shafts 3 and 6 are properly aligned with the two long axes. Such is facilitated because most of the remainder of the instrument will be absent due to its radiolucency. Examination of the radiograph should show that each pair of spaced apart bars appears as a single line; this assures that the correct alignment of the plane along which cutting will occur (which will be perpendicular to the centerline through the intermedullary (IM) canal of the radius) is being viewed.

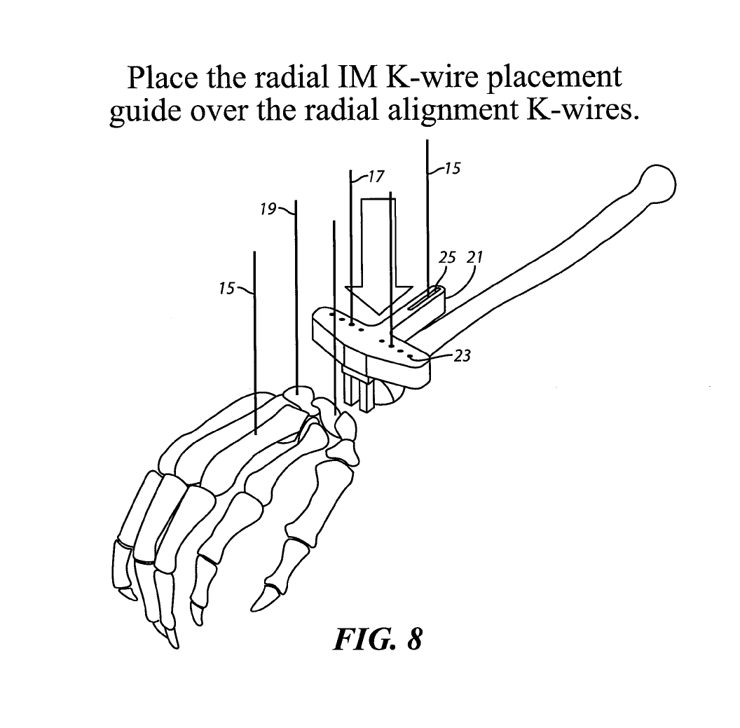

With the bone fragments from the resected ends removed, the surgical instrument is dorsally removed from the wrist by sliding it along the six parallel pins or K-wires which are seen in FIG. 7. Following its removal, an intermedullary placement guide 21 is attached to the resected radius using the three elongated pins 15 and 17 which are already seated in the radius, as shown in FIG. 8. The IM placement guide is generally T-shaped and includes a row of spaced apart holes 23 and an elongated slot 25 that duplicate the line of pinholes in the radial osteotomy block 1 and the elongated slot in the shaft 3 that accommodated the K-wire 15 that was driven through the saddle part 2.

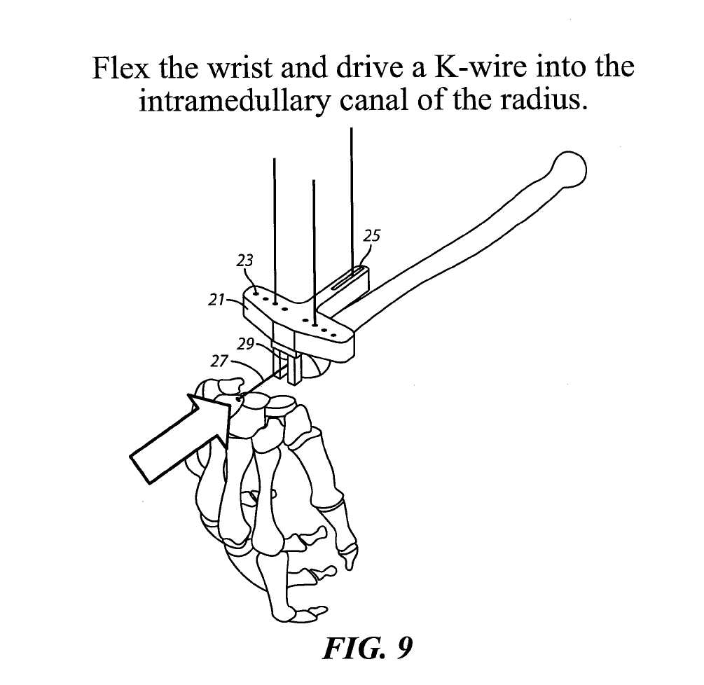

The three K-wires 15 and 19 that were driven into the carpal bone complex are then removed, and the wrist is placed in flexion to provide access to the resected distal end of the radius for broaching. With the wrist flexed, an elongated guide rod or K-wire 27 is driven into the IM canal of the radius through a guide opening 29 that is provided in the IM placement guide 21, as depicted in FIG. 9; the guide assures precise alignment within the IM canal. With this guide rod 27 in place, the placement guide 21 is removed dorsally, and the three K-wires 15 and 17 that were originally driven dorsally into the radius are also removed.

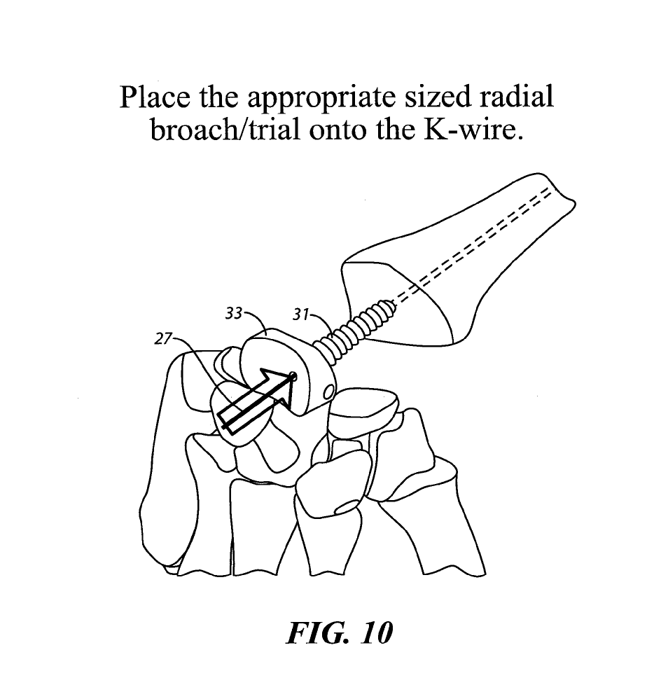

To broach the IM canal of the radius, a dual purpose, combined radial broach and trial implant 31 is selected which has the size of head 33 as was determined by reading the guide on the radial osteotomy block 1. As explained in more detail hereinafter, the combined broach/trial implants are provided in a desired variety of different head sizes, e.g. small (S), medium (M) and large (L) to accommodate patients with larger or smaller bone structures. The radial and carpal components are provided in pairs sized, e.g., S, M or L. Because greater amounts of bone resection may be required because of deterioration or the like, articular elements or inserts having slightly varying lengths are provided which correspond to each S, M or L pair. The appropriate elements to be used are shown by reading the markings along the lateral regions of the carpal osteotomy block (see FIG. 6) when all six of the K-wires were in place. With the chosen radial broach/trial implant placed on the guide rod 27, as depicted in FIG. 10, a radial impactor having an appropriately sized head is slid over the end of the guide rod, and the radial broach/trial implant is impacted into place in the distal end of the resected radius, maintaining its proper orientation until the flat surface of the head 33 of the radial trial implant is seated flush with the flat surface of the resected radius. The design of cutting teeth on the stem of this broach 31 is such that prior reaming of the IM canal is unnecessary. With this task accomplished, the elongated rod guide 27 that was inserted into the IM canal of the radius is removed distally.

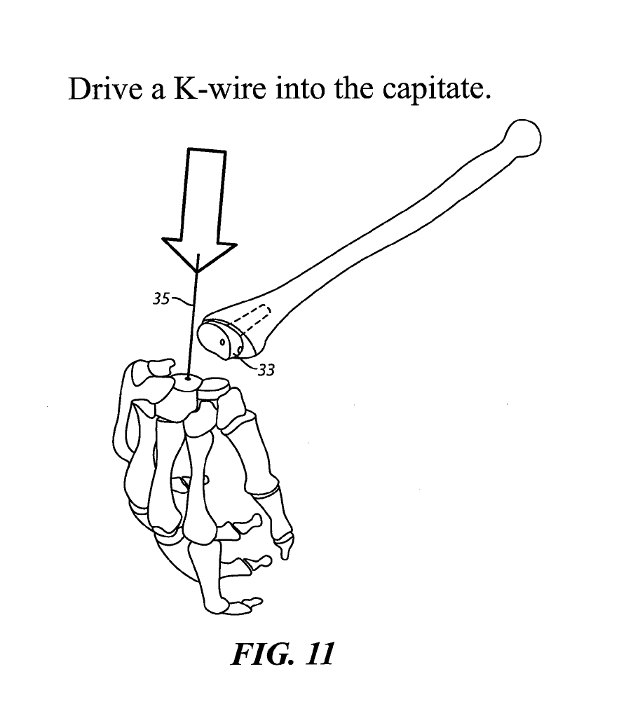

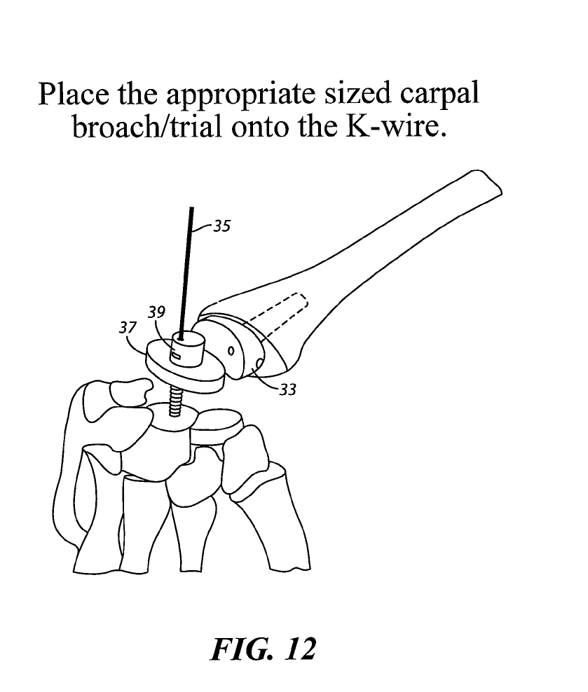

As depicted in FIG. 11, an elongated guide rod or K-wire 35 is driven into the IM canal of the capitate. With this elongated guide rod 35 in place, the appropriate-sized carpal broach/trial implant 37 (having a head which is a complement to that chosen relative to the distal end of the radius) is slid onto the guide as shown in FIG. 12. A carpal impactor is attached to the upstanding trunnion or post 39 of the carpal broach/trial implant, and it is impacted into place, maintaining proper orientation so as to align the lateral regions of the head of the implant 37 with the respective resected carpal bones. With the head of the implant 37 flush against the resected surface of the capitate, the carpal impactor is removed from the post 39, and the guide rod 35 is removed proximally. The seated carpal broach/trial implant is now ready to function as a carpal base plate in a range of motion (ROM) trial.

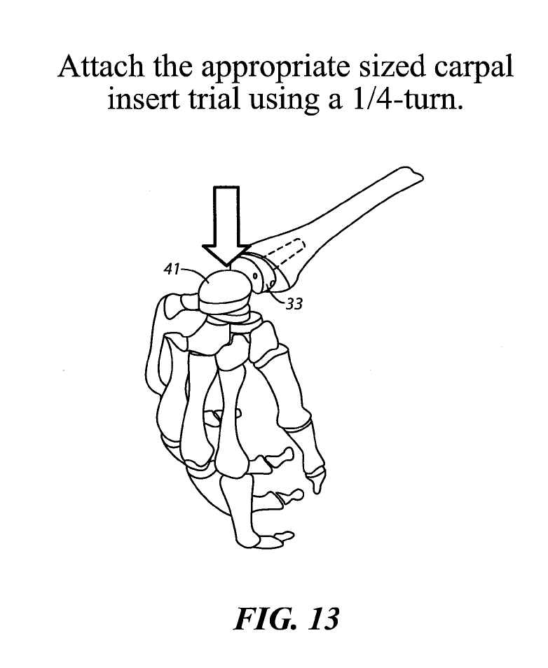

The appropriate-sized articular insert 41 is then placed over the post or trunnion 39, as depicted in FIG. 13, and then rotated 90° to interconnect it with the trial carpal implant 37. Selection is based upon the reading from the chart next to the opening adjacent the shoulder bolt heads 7, as mentioned above. The free distal surface of the articular insert 41 and the facing proximal surface of the head 33 of the radial trial implant lie in juxtaposition and are shaped so as to permit mutual smooth articulation movement in at least two different planes.

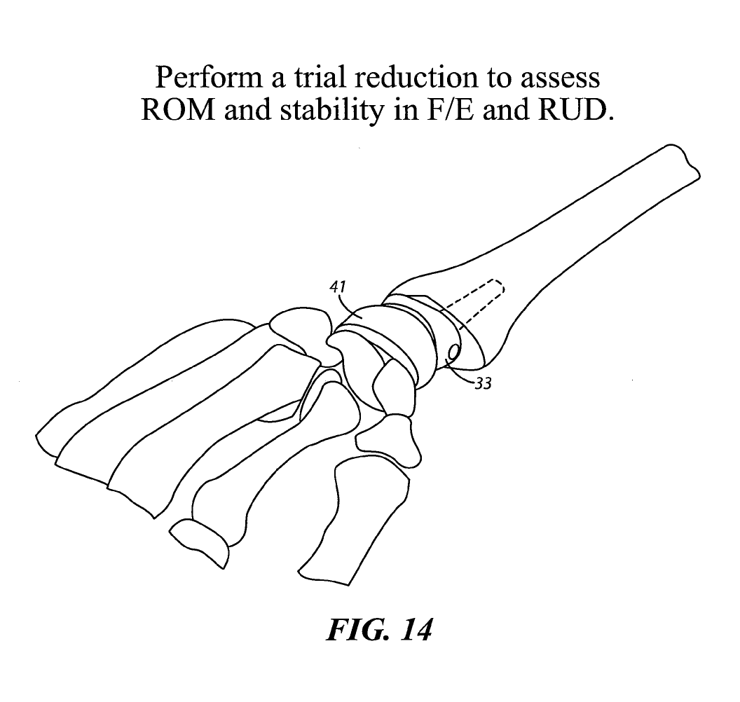

With the articular insert 41 in place, a trial reduction is performed to assess ROM and stability in flexion and extension, as well as radial and ulnar deviation (RUD), as depicted in FIG. 14. Assuming the trial components pass the test, the articulation insert 41 is removed using a quarter turn, i.e. 90° rotation. By reattaching the carpal impactor to the post 39, the carpal broach/trial implant 37 is readily removed. Extractor tongs are then used to distally remove the radial broach/trial implant 31, as by connecting through diametrically opposed recesses that may be optionally provided in the lateral rim of the head 33 of the radial trial implant. Following this removal, bone preparation is considered to be complete.

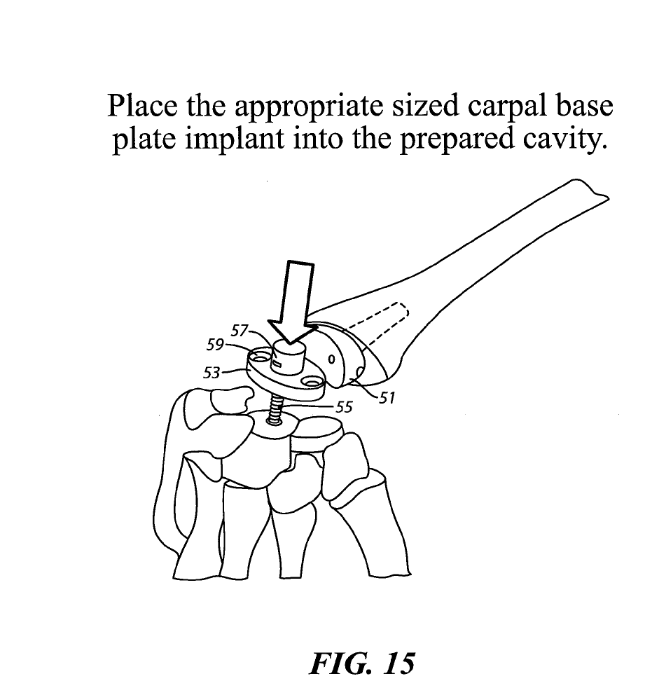

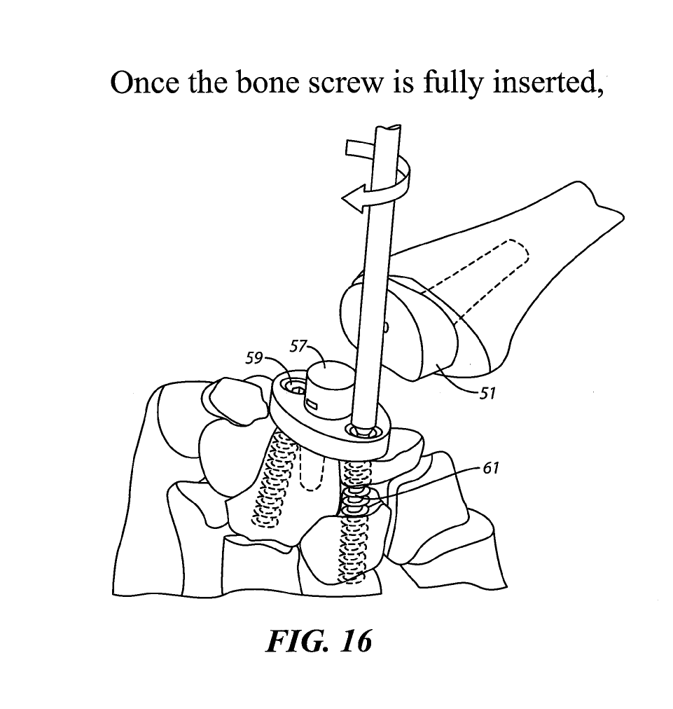

The stem of an appropriately sized radial component 51 (e.g., S, M or L) of a total wrist prosthesis is then inserted into the prepared cavity in the IM canal of the radius, and it is seated using a radial impactor in the same general manner as the trial implant 31 was inserted. The stem of the radial component 51 may be slightly greater in dimension than the stem of the respective trial implant to assure a tight fit is obtained. With the radial preparation complete, an appropriate-sized complementary carpal base plate component 53 is aligned with its stem 55 protruding into the open end of the broached IM canal of the capitate, as depicted in FIG. 15. The carpal impactor is then attached to its post 57, and the carpal component 53 is impacted into place so that its distal surface is flush with the resected flat surface of the capitate. Following removal of the carpal impactor, bone screw holes are prepared, as by drilling or the like through the pair of screw openings 59 in the base plate that flank the upstanding post and the oppositely extending stem (unless they were already prepared by drilling through screw openings that may be optionally provided in the carpal broach/trial implant before such was removed). Any suitable bone screws may be employed; for example, screws having spherical heads that will seat in spherical recesses formed in the base plate may be used. Screws 61 are then inserted as shown in FIG. 16.

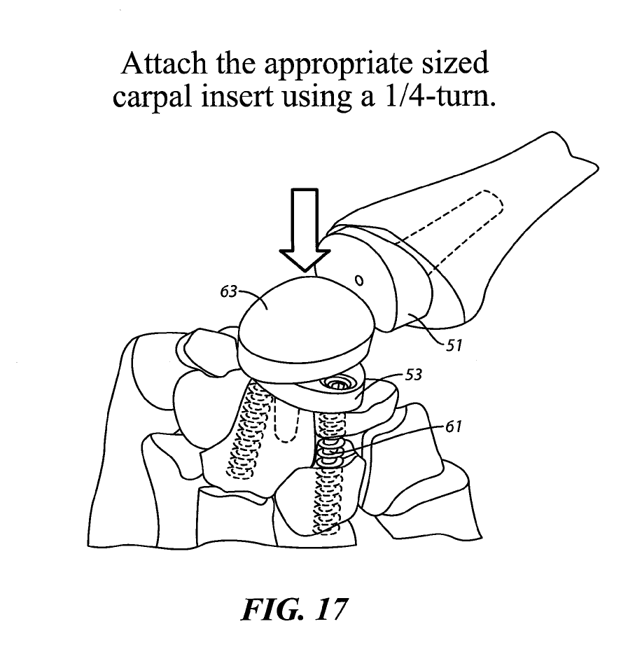

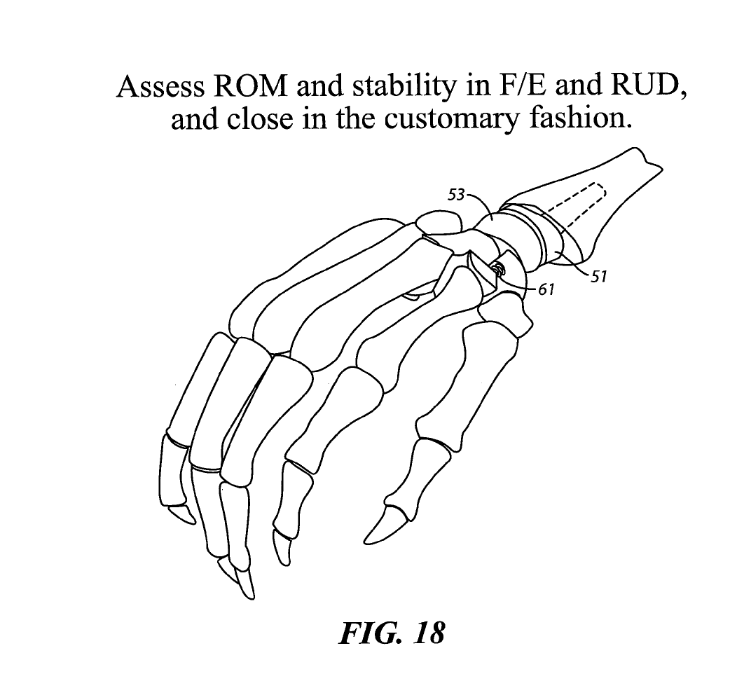

With both bone screws 61 in place in carpal bones which flank the capitate, such as the scaphoid and the hamate, a carpal articular insert 63 of complementary size and appropriate height (as was determined from the chart labeled on the carpal osteotomy block 4 adjacent the elongated opening for viewing the heads of the shoulder bolts 7) is attached to the base plate. It is inserted over the upstanding post or trunnion 57, as shown in FIG. 17. Then rotation of about ¼ turn) (90°) so that a pair of radially projecting lugs on the post 57 become entrapped axially within an interior groove formed in the articular insert secures it (see labeled FIG. 19) while the insert 63 remains free to rotate a few degrees in either direction, depending upon forces being applied through the articulating joint. With the articular insert 63 in place, the implantation is complete, and the surgeon can assess ROM stability in flexion and extension and RUD as depicted in FIG. 18. If satisfactory, the incisions are closed in the customary manner and fashion.

A more detailed explanation of the sizing of the components of the total wrist replacement prosthesis follows. As shown in FIGS. 4, 5 and 6, the radial osteotomy block 1 is positioned so that the cutting guide surface provided by the bars 10 is aligned to remove a minimal, but a complete, section of bone from the distal end of the radius. With this position acceptably defined, at least two K-wires 17 are inserted or drilled through two of the row of eight pinholes in the radial osteotomy block. At this time, the size of the radial component 51 that will be used can be determined by selecting one from among the, for example, three sizes that would likely be made available as part of a set of components for complete wrist replacement prostheses. For example, the radial components and the carpal components could each be provided in three different sizes, e.g. small, medium and large. The three radial components would be provided in both right and left sets; however, carpal components and articular inserts of complementary size would be the same for right and left wrists and would be based on the size of the radial component, e.g. all size “M” components would be used in one total wrist replacement. Each of the sets of articular inserts might be provided in three different heights. For example, the three medium articular inserts would all have bases that would mate with the medium carpal component base plate, i.e. the footprints would be the same. Generally, the size of the head of the radial component 57 that is chosen will be as large as possible without overhanging the resected end of the radius. The size of the head, i.e. S, M or L, would be noted, and the same size would be used for both the trial implant 31 and the ultimate radial component 51 of the prosthesis.

With the radial osteotomy block 1 thus secured in place by the three elongated rods 15 and 17, the surgeon may wish to fluoroscopically examine the surgical instrument and the wrist to decide how far it might be desirable to “open the window” between the two cutting guide surfaces. Again, the bones of the carpal bone complex would be viewed from an angle where the spaced apart upper and lower bars 11 that are embedded adjacent the volar and dorsal edges of the cutting guide surface are aligned, so the surgeon is certain he is viewing precisely along the plane that the resection will create. The elongated slot in the shaft 6 in the carpal osteotomy block 4 allows the block to be moved longitudinally while the saddle part 5 remains stationary in its location on the third metacarpal and the radial osteotomy block 1 is affixed dorsally to the radius. Once the approximate desired location is reached, the most appropriate spacer is selected. For example, spacers might be provided having widths of, e.g., 2.5 mm, 5 mm, 7.5 mm and 10 mm; along with simply flush alignment, such would provide for five different potential windows of spacing between the two cutting guide surfaces. The combined differences in longitudinal dimensions of the respective heads of the radial and carpal components of each set and the articular inserts of three different heights allow for the selection of a combination of heads and insert which will provide a prosthesis that will exactly fill the region in a patient's wrist that has been prepared by cutting along any of these five windows. The height of the articular insert 63 to be used when cutting was carried out along a specific window will be indicated by the chart labeled on the surface of the carpal osteotomy block adjacent the opening, as seen in FIG. 6.

Similar to such sets of radial components 51 and carpal components 53, each in three different sizes (with a different set of radial components for right wrists than for left wrists), the radial broach/trial implants 31 and carpal broach/trial implants 37 are similarly provided in sets of three, i.e. small, medium and large, where the heads are the same sizes as the respective heads in the sets of the final prostheses, along with nine articular inserts. The major differences in the components is that each dual purpose trial implant has a longitudinally extending passageway through which the elongated guide rod can pass and each has a stem which is shaped as a broach, being formed with a series of cutting edges that effect the reaming of the IM canal.

A representative wrist preparation kit would be provided that includes the surgical instrument, a set of three small, medium and large radial broach/trial implants 31 for a right wrist replacement, a similar set for a left wrist replacement, a set of three complementary, small, medium and large, carpal broach/trial implants 37, and a set of nine articular inserts 41, three each small, medium and large, which groups of different-sized articular inserts differ in height by 2.5 mm and by 5.0 mm. Also included would be impactors that might be used to seat the broaching stems of the dual purpose implants 31, 37 in the IM canals. A plurality elongated guide rods or K-wires that would be employed in the surgical operation would likewise be included.

From the foregoing, it should be apparent that the surgical instrument ensures that only a minimum amount of bone and cartilage is removed along the long axis of the radius and extending into the carpal bone complex, i.e. just the amount which is needed to accommodate the respective heads of the selected size total wrist replacement prosthesis. This is in contrast to many other such systems where cuts on the radial and carpal bones are made independently without precise regard to the total thickness of the prosthesis construct to be implanted; such can result in either joint laxity or in overstuffing.

Rather than utilizing certain anatomic landmarks within the carpal bone complex, e.g. the head of the capitate, to determine the extent of the resection, the surgical instrument references the cutting of the carpal bone complex precisely from the anticipated cut of the distal end of the radius. Moreover, the extent of the cut can be precisely adjusted by the surgeon after fluoroscopic examination of the precise cutting planes in prospective alignment with the bones of the carpal bone complex, before the last two K-wires 19 are driven into the carpal bones to set the final depth of the resection. Very importantly, this adjustment may be made after the plane of radial resection has been established. Such amount of cut is adjusted by changing the osteotomy window by the addition or changing of spacers. Excellent fluoroscopic visualization of the location of the radial and carpal osteotomy cutting planes is facilitated via the radiolucent materials from which the osteotomy blocks are formed, with only key elements being of radiopaque material. Thus, see-through visualization of all of the anatomic bony landmarks is allowed in conjunction with the pairs of radiopaque bars 10 and 11 that define the actual cutting guide planes.

In this respect, the use of two such spaced-apart rectilinear bars, e.g. of stainless steel, assures that the true osteotomy window is being viewed fluoroscopically when both the dorsal rail and the volar rail of one cutting guide surface are perfectly aligned and thus appear as one solid line in the fluoroscopic examination. Moreover, examination of the radio-opaque shafts 3 and 6, along which the saddle parts 2 and 5 slide, assures that the radial-ulnar position of the surgical instrument is properly aligned along the axes of the radius and the third metacarpal bone. Other radiopaque markers of varying lengths which are embedded in the radial osteotomy block 1, in the same dorsal-volar plane as the three longitudinal lines of different length that can be seen in FIG. 6 below the marks L, M and S, allow the surgeon to easily decide and note whether small, medium or large components are to be implanted, i.e., depending upon how these markers line up with the radial and ulnar edges of the radial eminence.

The concept of employing a single instrument to define both the cut to be made at the distal end of the radius and that of the carpal bone complex via a window in a single instrument reduces operating room time by avoiding the need to swap instruments. Moreover, the instrument is right/left reversible simply by rotating 180°, which is a further advantage in reducing the number of instruments required in a set for preparation of either a right or left wrist replacement.

One major further advantage lies in the concept of employing such dual purpose broaches/trial implants which not only reduces operating room time in avoiding the swapping of instruments, but minimizes potential damage to the precisely prepared bone cavity, as well as reducing the total number of instruments required for a complete set. This is a significant advantage over previous instrumentation where separate trial implants were uniformly used following independent broaching of the respective IM canal.

Although the invention has been described with regard to certain preferred embodiments, it should be understood that various modifications and changes as would be obvious to one having ordinary skill in this art may be made without departing from the scope of the invention, which is set forth by the claims appended hereto. For example, although the surgical instrument that is illustrated is designed so that the longitudinal spacing of the cutting guide surfaces provided by the window is effected by the insertion of separate spacers of different widths between the otherwise abutting surfaces of the two osteotomy blocks, the distance might alternatively be changed by the inclusion of spacers connected to one or both of the osteotomy blocks that could be rotated or slid into position to establish the desired spacing. As another alternative, detents might be employed that would engage cuts in the shoulder bolts at prescribed spaced intervals along the length thereof, or small pins might be inserted through one of, for example, four pinholes drilled in a shoulder bolt at precise, spaced apart intervals. Although this surgical instrument is illustrated for the resection of the radius and carpal bone complex to prepare for the implantation of a total wrist replacement prosthesis, it should be understood that its concept of providing a precise window of osteotomy for two bones of a joint to be replaced, as well as the use of dual purpose broach and trial implants, could be advantageously employed for resection and/or replacement of other joints in the human body.

Particular features of the invention are emphasized in the claims which follow.

1. A surgical instrument for mounting dorsal of a patient's wrist to guide resection of the radius and the carpal bone complex during preparation for implanting a radial component and a carpal component as part of a total wrist replacement, which surgical instrument comprises:
(a) a radial osteotomy block having a cutting guide surface,
(b) a carpal osteotomy block having a cutting guide surface,
(c) means linking said osteotomy blocks in generally planar orientation so that said blocks can move longitudinally relative to each other to alter the separation distance between said cutting guide surfaces of said blocks,
(d) means for attaching said instrument to the radius and a carpal bone of a patient in a manner so said osteotomy blocks remain longitudinally movable relative to these bones,
(e) means for adjusting the longitudinal distance between said cutting guide surfaces to facilitate osteotomy of the patient's wrist to the desired degree, and
(f) means for stably attaching said blocks to bones at the wrist of a patient with said cutting guide surfaces spaced the desired distance apart.

2. The instrument of claim 1 wherein each of said blocks has a longitudinally aligned shaft on which a saddle part is slidably mounted to provide said attaching means, with each of said saddle parts being disposed in an opening in one said respective block so that there can be relative motion between said saddle part and the remainder of said block when said saddle part is connected to either the radius or the carpal bone.

3. The instrument of claim 2 wherein said saddle parts include oppositely facing upper and lower concave saddle surfaces and wherein said instrument can be aligned with either a right or left wrist by inverting it.

4. The instrument of claim 1 wherein said linking means effect slidable mounting of at least one of said blocks in interconnection with the other, along a pair of parallel spaced apart axes, and means is provided for resiliently biasing said blocks toward each other along said two axes.

5. The instrument of claim 4 wherein said instrument includes a pair of parallel rods that are mounted in said radial osteotomy block and that are laterally offset from each other and that define said axes, along which pair of rods said carpal osteotomy block is slidably movable to effect such adjustment in distance between said cutting guide surfaces.

6. The instrument of claim 5 wherein a plurality of spacer elements of different widths are provided for varying the longitudinal distance between the spaced apart cutting guide surfaces in the instrument when mounted on a patient's wrist.

7. The instrument of claim 1 wherein said carpal osteotomy block has size markers adjacent an elongated aperture through which the location of the head of at least one said rod may be viewed, which location is indicative of the longitudinal distance between said cutting guide surfaces.

8. The instrument of claim 1 wherein said blocks are generally radiolucent but each includes a pair of rectilinear, parallel, radiopaque bars mounted at the dorsal and volar surfaces of the cutting guide surface, which rectilinear bars are spaced apart from each other, and wherein said parallel shafts are also made of radiopaque material.

9. The instrument of claim 8 wherein said radial osteotomy block also includes radiopaque markers which are located to represent the size of heads of different-sized radial components that are available for implantation so selection of the size of a radial component can be made.

10. A surgical kit for preparing the wrist of a patient for implantation of a total wrist prosthesis, which kit comprises:
(a) a surgical instrument for mounting dorsal of a patient's wrist, which instrument includes:
a radial osteotomy block having a cutting guide surface,
a carpal osteotomy block having a cutting guide surface,
means linking said osteotomy blocks in generally planar orientation so that said blocks can move longitudinally relative to each other to alter the separation distance between said cutting guide surfaces of said blocks,
means for attaching said instrument to the radius and to one carpal bone of a patient in a manner so that said osteotomy blocks can be moved distally-proximally relative to said bones,
means for adjusting the longitudinal distance between said cutting guide surfaces to facilitate osteotomy of the patient's wrist to the desired degree while said instrument is so attached, and
means for stably affixing said instrument to bones at the wrist of a patient with said cutting guide surfaces are spaced the desired distance apart;
(b) a plurality of radial broach/trial implants each having a head with a concave surface and a broaching stem, the heads of which radial implants range in size;
(c) a plurality of carpal broach/trial implants each having a head with a protruding interconnector and a broaching stem, the heads of which carpal implants range in size; and
(d) at least one articular insert for connection with said carpal broach/trial implant interconnector, which insert has a convex surface of a curvature matched to articulate with said concave surface of one said radial broach/trial implant.

11. A method for the implantation of a total wrist replacement, which method comprises the steps of:
(a) attaching a surgical instrument dorsal of the patient's wrist by initially attaching said instrument to the radius and to a carpal bone, said instrument comprising a radial osteotomy block and a carpal osteotomy block which are longitudinally movable relative to each other, each of which blocks has a cutting guide surface;
(b) adjusting said radial osteotomy block proximally/distally relative to the distal end of the radius and securing said radial osteotomy block in said adjusted location;
(c) positioning said carpal osteotomy block at a desired location where its cutting guide surface is at a desired distance from the cutting guide surface of said radial block and is aligned with bones of the carpal bone complex to be resected, and securing said carpal block in such location;
(d) respectively cutting the radius and bones of the carpal bone complex along planes defined by said two cutting guide surfaces of said radial and carpal osteotomy blocks;
(e) removing said surgical instrument from the wrist; and
(f) broaching the resected radius and one resected carpal bone preparatory to installing a total wrist replacement.

12. The method of claim 11 wherein, following said removal step (e), the radius is broached through the resected end thereof by inserting a radial broach/trial implant therein;
the capitate which is one of the resected carpal bones is broached by inserting a carpal broach/trial implant into the resected end thereof;
a trial articular element is attached to said carpal broach/trial implant;
a trial reduction is performed to assess range of motion (ROM);
said trial element and trial implants are removed; and
a radial component, a carpal component and an articular element are installed to provide a total wrist replacement.

13. The method of claim 11 wherein said adjusting of said radial osteotomy block is carried out by its longitudinal sliding movement relative to parts of the instrument which are attached to the radius and to the carpal bone.

14. The method of claim 13 wherein said carpal osteotomy block is secured in a desired position by spacing said carpal block a desired distance from said radial block.

15. The method of claim 13 wherein said radial osteotomy block is secured in said adjusted location by inserting at least two elongated pins through holes in said block so said pins seat in the radius of the patient.

16. The method of claim 15 wherein said carpal osteotomy block is secured by inserting elongated pins through holes in said carpal block so said pins seat in bones of the carpal bone complex.

17. The method of claim 15 wherein, following said cutting of step (d) and said removal of the instrument from the patient's wrist in step (e), an intermedullary placement guide is attached to the radius using said elongated pins which were seated in the radius in order to provide a guide through which to insert an elongated rod into the intramedullary canal of the radius to facilitate said broaching.

18. The method of claim 17 wherein an elongated guide rod is driven into the intermedullary canal of the radius through a guide opening provided in said placement guide and said placement guide is then removed and wherein a radial broach/trial implant, which has a central longitudinal passageway, is inserted along said guide rod to simultaneously broach said radius and insert a stem portion on said trial implant.

19. The method of claim 18 wherein an elongated guide rod is driven into the resected end of the central one of the carpal bones resected, and wherein the stem of a carpal broach/trial implant is slidably inserted along such guide rod to simultaneously broach the intermedullary canal thereof and insert a stem portion of said trial implant and wherein a carpal component, when later installed, is connected to the capitate of the carpal bone complex through a central stem portion and is secured via fasteners inserted into other bones of the carpal bone complex through holes in a head portion of said carpal component which flank said stem.

20. (canceled)

21. The method of claim 11 wherein said surgical instrument is initially attached by passing first and second elongated pins through holes in two saddle parts that respectively rest dorsally on the radius and on the third metacarpal of the patient, which saddle parts are separately mounted on two parallel rods of said instrument having slotted passageways provided therein through which said first and second pins extend.

22. (canceled)
